(19) United States
(12) Patent Application Publication
(10) Pub. No.: US 2021/0213224 A1
(43) Pub. Date: Jul. 15, 2021

(54) TRACHEAL CANNULATION DEVICE

(71) Applicants: Steven VENTICINQUE, San Antonio, TX (US); Justin RICE, San Antonio, TX (US); Travis CHASTAIN, San Antonio, TX (US)

(72) Inventors: Steven VENTICINQUE, San Antonio, TX (US); Justin RICE, San Antonio, TX (US); Travis CHASTAIN, San Antonio, TX (US)

(21) Appl. No.: 17/055,530

(22) PCT Filed: May 14, 2019
(86) PCT No.: PCT/US2019/032193

§ 371 Date: Nov. 13, 2020

Related U.S. Application Data
(60) Provisional application No. 62/752,255, filed on Oct. 29, 2018.
(60) Provisional application No. 62/671,104, filed on May 14, 2018.

(51) Int. Cl. A61M 16/04; A61M 25/01

(52) U.S. Cl. CPC A61M 16/0488; A61M 2205/0266; A61M 25/0102; A61M 16/0418

(57) ABSTRACT

Certain embodiments are directed to a bougie device and methods of use thereof, the device directing the deployment of an endotracheal tube through the larynx.

### CROSS-REFERENCE TO RELATED APPLICATIONS

This Application claims priority to U.S. Provisional Applications 62/671,104 filed May 14, 2018 and 62/752,255 filed Oct. 29, 2018, each of which is incorporated herein by reference in its entirety.

### BACKGROUND

Certain embodiments are directed to the field of medicine. In particular certain embodiments are directed to tracheal intubation devices.

Laryngoscopy can be used to assist tracheal intubation and involves the insertion of a laryngoscope to facilitate the visualization of the vocal cords (the visualization phase of tracheal intubation). This is followed by the insertion of an endotracheal tube (ETT) through the vocal cords (glottis) and then downward into the trachea (referred to as the insertion and cannulation phases of tracheal intubation, respectively). Traditionally, direct laryngoscopy (DL) has been employed to expose the glottis so that operators can view it directly in order to insert an ETT. A metallic stylet is usually placed within the ETT to promote rigidity and malleability in order to ease insertion. The stylet is more rigid than the ETT and will maintain its shape under normal loading conditions. Occasionally, during DL operators are faced with patients having anatomic features that make visualization of the vocal cords difficult or impossible. These circumstances, along with advances in fiber-optic and digital camera technology, have led to the development of video laryngoscopy (VL) which employs laryngoscopes with distal cameras or fiber-optic bundles. VL's make viewing the glottic aperture easier despite occasional challenging anatomic conditions or lower operator laryngoscopy skill levels. As a consequence, even novice operators can now almost always visualize the vocal cords in circumstances where it would have been previously difficult. DL and VL are both used in day-to-day tracheal intubation practice.

VL continues to emerge as an increasingly accessible and utilized technique to expose the glottis during tracheal intubation, particularly in circumstances of anticipated difficulty and during emergency airway management outside of the operating room. Although VL has made visualizing the vocal cords easier, a new challenge has emerged. During DL the path to ETT insertion is essentially a straight axis because the operator exposes the route via direct vision and aligns the oral, laryngeal, and tracheal axis. This usually requires a minimal bend of the distal portion of the stylet and ETT. However, with VL the cords are exposed with a camera or fiber-optic bundle located near the tip of the laryngoscope. The resulting approach to the vocal cords can be up to 90 degrees offset from the ETT axis of insertion at the mouth, in contradistinction DL, where the oral, laryngeal, and tracheal axis are nearly aligned during insertion and tracheal cannulation. Video laryngoscopes, particularly those with hyperangulated blades require a substantial curvature of the ETT and stylet. This offset angle and the additional skill required to insert the ETT via an indirect, 2-dimensional video screen view of the vocal cords makes ETT insertion into the glottis more difficult (the insertion phase). Furthermore, the resultant angle of the naturally downward angled trachea and the incoming ETT can cause the ETT to collide with the anterior portion of the trachea impeding ETT advancement into the trachea (the cannulation phase). Poor exposure of the glottic aperture with the video laryngoscope can exacerbate the problem. This difficulty cannulating the trachea with the ETT when using VL is a well described phenomenon.

Operators can find themselves with an adequate view of the vocal cords, the ETT engaged within the glottic aperture, yet are unable to advance the ETT into the trachea. This can occur despite the use of rigid stylets designed specifically to accommodate the angle required to facilitate VL intubation. Several methods have been described to overcome this problem, including the placement of a bougie into the trachea using VL, and then railroading the ETT over the bougie into the trachea—as used herein a bougie is a thin cylinder of rubber, plastic, metal or other material that is inserted into or through a body passageway, such as the esophagus, to diagnose or treat a condition, and can be used to widen a passageway, guide another instrument into a passageway, or dislodge an object. One method used to overcome failed advancement of the ETT into the trachea is to leave the ETT engaged in the glottis, remove the stylet, and advance a bougie down the ETT and into the trachea, then railroad the ETT over the bougie. Another is to pull back the stylet and clockwise rotate and advance the ETT so it's curvature is more aligned with tracheal descent angle. However, these methods can be challenging, especially for less-skilled operators. These difficulties can lead to multiple intubation attempts which can result in laryngeal injury and increased complications. Overt intubation failure can result in death or brain damage. A number of studies have correlated the increased risk of adverse events associated with multiple intubation attempts (Mort, Anesthesia and Analgesia. 2004; 99:607-13).

There remains a need for additional devices to better facilitate the insertion of ETTs.

### SUMMARY

The device described herein provides a scientific, anatomic-based solution to a well characterized clinical problem (the failure of tracheal intubation due to glottic insertion and tracheal cannulation issues). Currently, no devices approach this problem in a similar manner or with a similar design providing for directing an integrated bougie smoothly down the trachea avoiding or minimizing collision into the anterior tracheal rings and then cannulating the trachea with the ETT down the integrated bougie guide continuously using primarily one hand.

Certain embodiments are directed to a tracheal cannulation device comprising: a curved, stylet comprising a proximal end and distal end; a flexible bougie positioned in the lumen of a hollow stylet or surrounding a solid, wire stylet; a handle attached to the proximal end of the stylet, the handle having (i) a handle core portion forming a lumen or opening for a stylet or bougie to traverse the handle and at least two stop portions, finger rests, or finger rings projecting from the handle core portion away from the long axis of the device, a first (posterior) stop portion or finger rest possessing a thumb rest position in top of the stop portion or finger rest, (ii) an endotracheal tube (ETT) advancer portion having a proximal thumb tab configured to provide for applying force along the axis of the ETT advancer and ETT, and a distal ETT collar configured to hold and provide for advancement of the proximal end (connector) of the ETT, (iii) a bougie advancer portion having a proximal thumb button coupled to the flexible bougie configured to apply force to the bougie along the long axis of the bougie and bougie advancer; and a rigid hypotube traversing the handle and forming a lumen in which or below which the bougie is positioned and to which the stylet is attached or being solid and attaching to a flexible bougie distally. The ETT advancer portion can be an adjustable two-piece ETT advancer portion and can be made from metal or thermoplastic polymer or a combination thereof. The handle can further include a locking mechanism to reversibly secure the ETT advancer to the handle core. In certain aspects, the locking mechanism is a detent or ratchet mechanism. The device can further include a hollow endotracheal tube (ETT), wherein at least a portion of the stylet and/or bougie is capable of being contained within the ETT, and wherein the ETT is capable of being extended past the distal end of the stylet. The stylet can have a curve that is a distal curve with an angle of between 20, 30, 40, 50 and/to 60, 70, 80, 90 degrees, including all values and ranges there between. The hypotube can be metal, thermoplastic polymer, or combinations of metal and thermoplastic polymer. The stylet curve can be a distal curve with an angle of between 10 and 90 degrees. The device can be configured to be used for the tracheal intubation of a humans or non-human mammals.

Embodiments of the invention are directed to a design/mechanism that directs a bougie downward into the trachea during its deployment in order to minimize or avoid collision of the bougie into the anterior trachea. Dynamic directionality of the bougie can be accomplished using a Nitinol, or similar shape memory material, wire or strip. Nitinol is a shape-maintaining metal, so when the bougie is deployed it bends the distal portion downward.

Another feature can be a bougie-over-stylet design. Here, instead of the bougie being housed within a hollow stylet, a solid inner wire is present with a hollow bougie covering the wire. This allows a larger dimeter bougie. One aspect of this design is the manner in which the inner wire connects to the handle. The inner wire can have a bend or attachment portion forming and angle with respect to the long axis. In certain aspects the angle is about 90 degrees. The attachment portion of the inner wire can be coupled to the handle or a hypotube. In certain instances the handle or hypotube will have notch to receive the inner wire attachment portion and secure it to the handle assembly.

In certain embodiments the bougie tip will be soft and atraumatic.

Certain embodiments are directed to a bougie comprising a proximal end and distal end, wherein the proximal end is configured to be coupled to a deployment device or handle (e.g., an ETT deployment device), and the distal end comprising a rounded tip configured to deploy into the trachea of a subject; and the bougie having an inner guide that comprises a shape memory portion that returns to a default geometry that curves away from the curve of a deployment device when the bougie is deployed during use. The shape memory portion can be 1, 2, 3, 4, 5, 6, 7, 8, 9, 10, 11, 12, 13, 14, 15, 16, 17 to 18 mm in length, including all values and ranges there between. The shape memory portion can be titanium, nickel, nitinol, stainless steel alloys, niobium, zirconium, cobalt-chrome alloys, molybdenum alloys, tungsten-rhenium alloys and any combination thereof. In certain aspects the shape memory portion is nitinol. The shape memory portion or guide can be inside an exterior outer coating that forms the surface of the bougie.

In other embodiments a bougie can comprise a proximal end and distal end, wherein the proximal end is configured to be coupled to a deployment device, and the distal end comprising a rounded tip. The bougie can have at least two lumens, (a) a first lumen containing or configured to contain an inner stylet wherein the bougie is configured to extend beyond the stylet, and (b) a second lumen containing a shape memory guide that is configured to extend with the bougie, the shape memory guide comprising a shape memory portion that returns to a default geometry that curves away from the curve of the stylet when the bougie is extended beyond the stylet. The shape memory portion is titanium, nickel, nitinol, stainless steel alloys, niobium, zirconium, cobalt-chrome alloys, molybdenum alloys, tungsten-rhenium alloys and any combination thereof. In certain aspects the shape memory portion is nitinol. The shape memory guide can be a wire or a strip. A strip being substantially rectangular or oval in cross section (having a width longer than the height) forming a flattened strip. The stylet can be a rigid stylet. The stylet can deform the shape memory guide from its default geometry. The stylet can be an aluminum, stainless-steel, or another semi-rigid material. In certain aspects the stylet is a curve with an angel of between 10 and 90 degrees. In other aspects the stylet is capable of being bent by a user and retain its shape during use of the device during a tracheal intubation procedure. Any embodiment of a bougie described herein can further comprise a hollow endotracheal tube (ETT), wherein at least a portion of the bougie is capable of being contained within the ETT, and wherein the ETT is capable of being extended past the distal end of the bougie.

In another embodiment a bougie can have a proximal end and distal end, wherein the proximal end is configured to be coupled to a deployment device, and the distal end comprising a rounded tip configured to deploy into the trachea of a subject, wherein the bougie does not contain a shape memory guide but has a distal shape and/or hardness that allow it to deflect off of the anterior tracheal wall and progress inferiorly down the trachea during deployment.

Certain embodiments are directed to methods for tracheal intubation of a subject having a glottic aperture, vocal cords, and a trachea using any bougie as described herein. In certain aspects the method includes obtaining any bougie described herein with an ETT loaded thereon; placing the distal end of the bougie into and/or directly in front of the glottis of the subject; extending the bougie past the distal end of the ETT, below the glottis of the subject, and into the trachea; extending the ETT past the distal end of the bougie, through or below the vocal cords of the subject, and into the trachea; and removing the bougie from the ETT. The subject can be a human or non-human mammal.

Other embodiments are directed to methods for the tracheal intubation of a subject having a glottis and a trachea comprising: obtaining a bougie of claim 6 with an endotracheal tube (ETT) loaded thereon; placing the distal end of the ETT/bougie into and/or directly in front of the glottis of the subject; advancing the ETT/bougie simultaneously through the glottis where the memory guide-shaped bougie bends the ETT posteriorly in order to guide it away from the anterior trachea so it can easily descend inferiorly; and removing the bougie from the ETT once the ETT is positioned in the trachea. The subject can be a human or non-human mammal.

In other embodiments the devices described herein can be incorporated into a sterile cover or kit.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 is a front perspective view of tracheal cannulation device assembly showing the handle portion engaged with an ETT.

FIG. 2 is a backside view of tracheal cannulation device assembly showing the handle portion engaged with an ETT.

FIG. 3 is a top view of tracheal cannulation device assembly showing the handle portion engaged with an ETT.

FIG. 4 is an enlarged view of the handle.

FIG. 5 is an exploded view of one embodiment of the handle/ETT assembly.

FIG. 6 is multiple views of one embodiment of the an ETT advancer.

FIG. 7 is multiple views of one embodiment of the an ETT advancer engaging an ETT.

FIG. 8 is multiple views of one embodiment of the an ETT advancer with a locking mechanism (e.g., detent) engaging an ETT.

FIG. 9 shows multiple views of a handle assembly with a hypotube and stylet mechanism.

FIG. 10 shows multiple views of a hypotube/stylet assembly.

FIGS. 11A and 11B illustrate components of Design 2.

FIGS. 12A and 12B illustrate components of Design 4.

FIG. 13 illustrates Design 1.

FIG. 14 illustrates the operation of Design 2.

FIG. 15 further illustrates operation of Design 2.

FIG. 16 illustrates operation of Design 3.

FIG. 17 illustrates operation of Design 4.

FIG. 18 (A) illustrates an example of one embodiment where the outer diameter of the bougie and the inner diameter of the ETT are such that the gap between the two is minimized. Also illustrated is an example of two embodiments of the bougie, one embodiment having a circular cylinder shaped bougie and a second embodiment having a star cylinder shaped, both bougie being a double lumen bougie providing for an inner wire and a memory wire. (B) illustrates two separate configurations for a dual lumen bougie. A first configuration with the inner wire and memory wire running substantially the length of the bougie or a second configuration with inner wire at an intermediate length and the memory wire extended to the end of the bougie, both of which can be capped with a soft tip.

FIG. 19 illustrates the side views of one embodiment.

FIG. 20 illustrates perspective views of the embodiment of FIG. 19.

FIG. 21 illustrates two embodiments of an exploded view of the embodiment of FIG. 19 and FIG. 20. (A) Is a size adjustable assembly with an adjustable two piece ETT advancer and (B) is a single size assembly with a one piece ETT advancer.

FIGS. 22A-22C illustrates certain steps in the operation of an embodiment illustrated in FIG. 19, FIG. 20, and FIG. 21.

### DESCRIPTION

Yearly millions of people undergo tracheal intubation. Most tracheal intubations are performed in operating rooms by anesthesiologists or nurse anesthetists. Direct laryngoscopy (DL) is likely the most prominent technique. However, the routine use of video laryngoscopy is increasing rapidly, as VL is now present in nearly every setting where tracheal intubation occurs. The advent of VL has created new challenges that include occasional difficulty advancing the ETT into the trachea during the procedure. The ETT approach to the trachea during VL can be up to 90 degrees from axis of insertion at the mouth. The resulting approach to the vocal cords can be up to 90 degrees offset from the ETT axis of insertion at the mouth, in contradistinction DL, where the oral, laryngeal, and tracheal axis are nearly aligned during insertion and tracheal cannulation. Video laryngoscopes, particularly those with hyperangulated blades require a substantial curvature of the ETT and stylet. This offset angle and the additional skill required to insert the ETT via an indirect, 2-dimensional video screen view of the vocal cords makes ETT insertion into the glottis more difficult (the insertion phase). Furthermore, the resultant angle of the naturally downward angled trachea and the incoming ETT can cause the ETT to collide with the anterior portion of the trachea impeding ETT advancement into the trachea (the cannulation phase). A poor exposure of the glottic aperture with the video laryngoscope can exacerbate the problem. This difficulty cannulating the trachea with the ETT when using VL is a well described phenomenon. In some instances, the device described herein has a curved, hollow stylet or solid stylet that can incorporate an extending or telescoping bougie in the lumen or around the exterior of the stylet or otherwise coupled to a stylet as described herein. In some instances, this will allow the operator to place the ETT and stylet above or through the glottis, advance the narrow, integrated bougie through vocal cords, then advance the ETT over the bougie into the trachea. In some instances, the stylet provides the appropriate curvature in order to engage the glottic aperture or glottis during VL, and the bougie provides proper (tracheal) directionality for the ETT while axial force is applied to the ETT by the operator above. The bougie also directs the ETT downward and away from the anterior larynx where it can sometimes collide and hang up.

### I. Intubation Devices

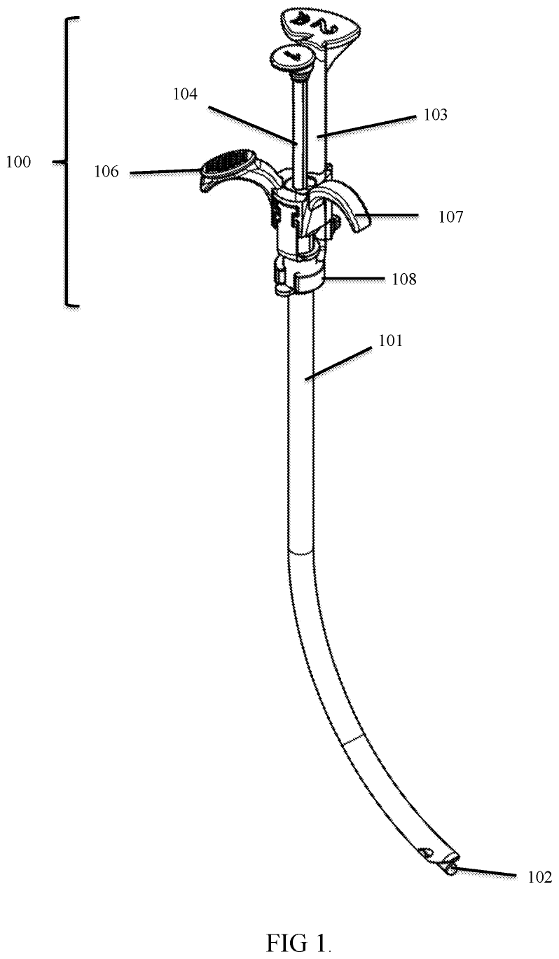

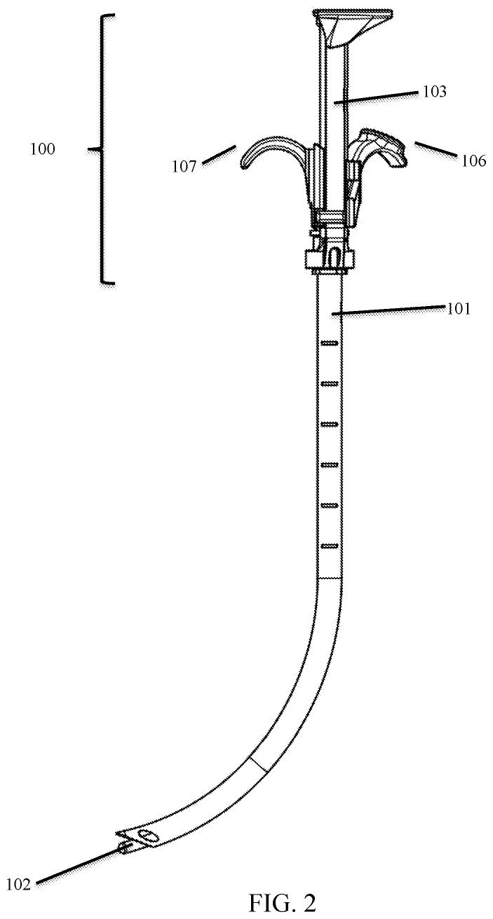

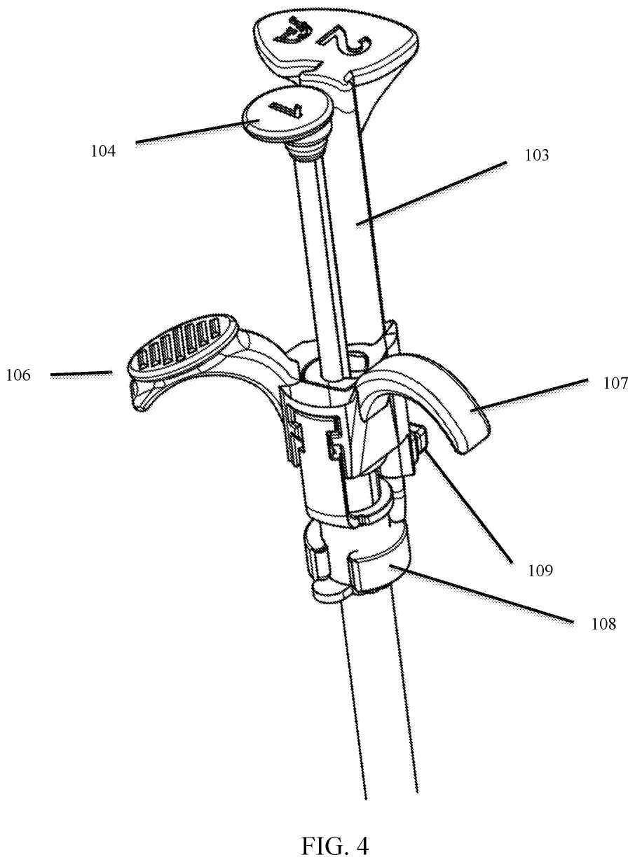

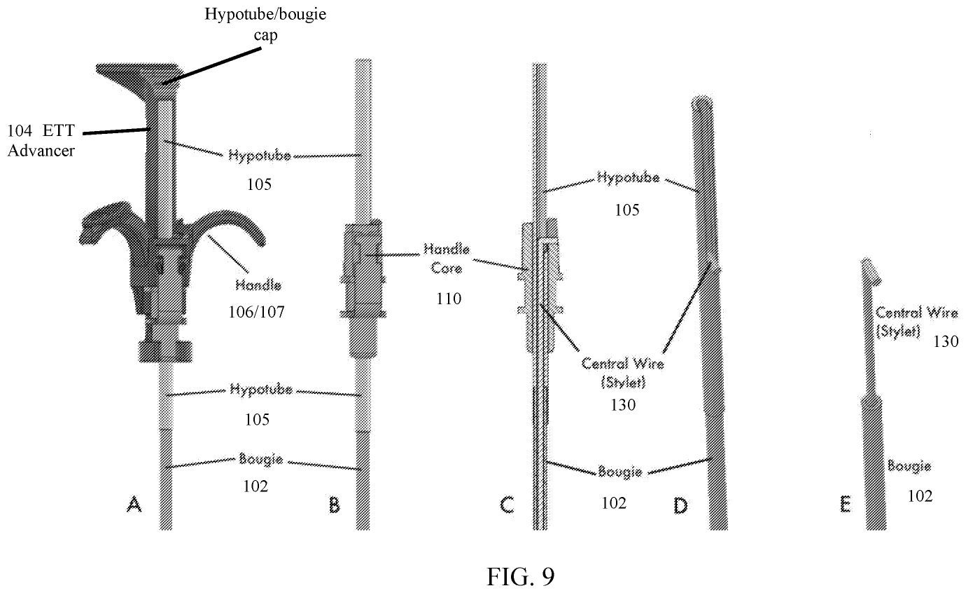

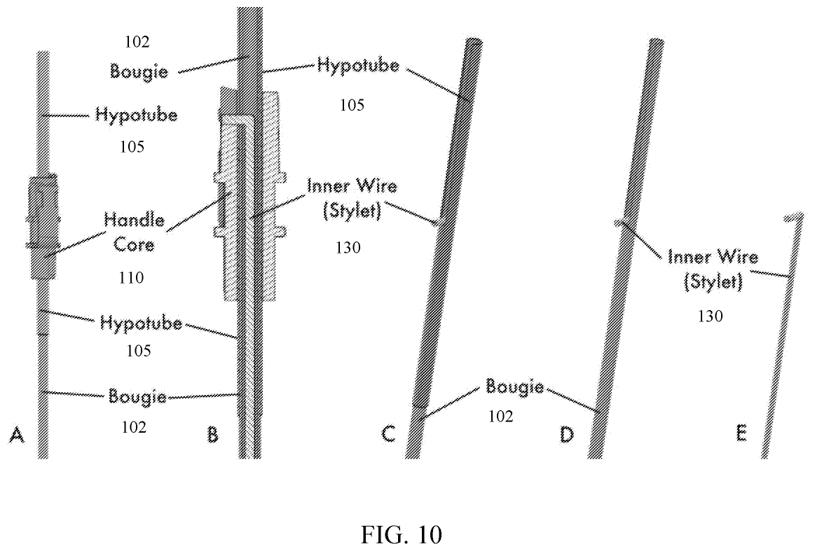

In certain embodiments, a newly described handle allows operators to perform the insertion phase (ETT inserted through the vocal cords) by either holding the central portion of the ETT or the handle (FIG. 1 and FIG. 2). The handle can comprise an ETT advancer portion 103 (having a distal ETT collar portion 108 that engages the ETT connector), a bougie advancer portion 104 (operatively coupled to a bougie, stylet, or bougie and stylet assembly that can be positioned in the lumen of an ETT), a hypotube portion 105 (See FIG. 9 and FIG. 10), at least two stop portions or finger rests—a first stop portion or finger rests with a thumb rest 106 (See FIG. 4) and a second stop portion 107. It also allows an easy hand transition from either hold so the operator can perform the cannulation phase (bougie deployment followed by ETT advancement). This handle configuration also allows the operator to reach higher above the handle in order to actuate both the bougie and the ETT advancer component. As a consequence, a previous two-tab advancer design can be eliminated and just use one tab is needed as a means to adequately advance the ETT into the trachea.

FIG. 1 and FIG. 2 shows an example of a newly designed handle with the bougie-over-wire device assembly. FIG. 1 shows handle mechanism 100 engaged with ETT 101 having bougie 102 positioned in the lumen of ETT 101. This design allows the operator to hold ETT or handle during insertion. FIG. 2 shows a backside view of handle mechanism 100 engaged with ETT 101 having bougie 102 positioned in the lumen of ETT 101. This design allows the operator to hold ETT or handle during insertion. Operator can more easily actuate the bougie 104 and ETT advancer 103. This allows elimination of a bottom thumb tab of the ETT advancer. The design is also lower profile, allowing easier packaging and lower manufacturing costs. In certain instances the operator can use stop portion/finger rest to assist in advancement of the ETT advancer or bougie advancer by engaging the stop portion/finger rest 106/107 with the fingers, much like a hypodermic needle, to provide counter force to thumb applied force.

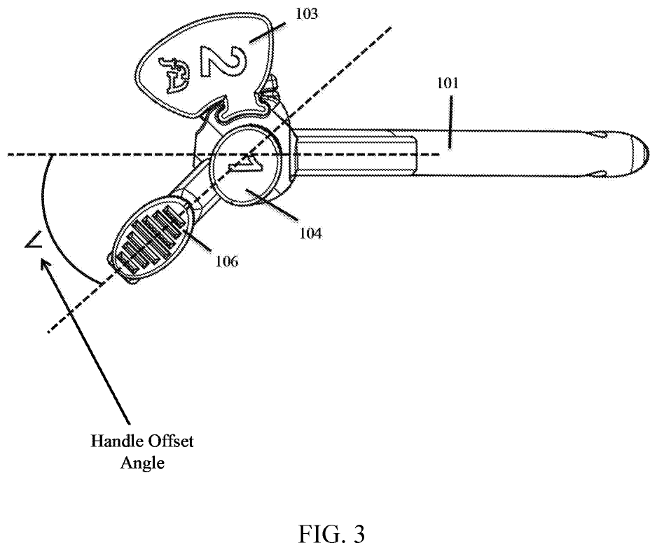

FIG. 3 shows a top view of the handle showing ETT advancer portion 103, a bougie advancer portion 104, a first stop portion with a thumb rest 106 and ETT 101 visible due to curvature. The posterior stop portion or finger rest that incorporates the thumb rest 106 can be offset from the axis of anterior stop portion or finger rest by 30 to 50 degrees. This allows a more ergonomic right hand and right arm positioning by the operator during the tracheal intubation procedure.

FIG. 4 is an enlarged view of the handle having ETT advancer portion 103 (having a distal ETT collar portion 108 that engages the ETT connector and holds the connector in a manner whereby the ETT will not prematurely advance during bougie deployment), a bougie advancer portion 104 with a proximal button (operatively coupled to a bougie, stylet, or bougie and stylet assembly that can be positioned in the lumen of an ETT), a hypotube portion 105 (See FIG. 9 and FIG. 10), at least two stop portions or finger rests—a first stop portion with a thumb rest 106 and a second stop portion 107. FIG. 4. Also provides a view of one embodiment comprising locking mechanism 109, which can be in the form of a detent or other mechanism that can reversible lock the ETT advancer at a selected position.

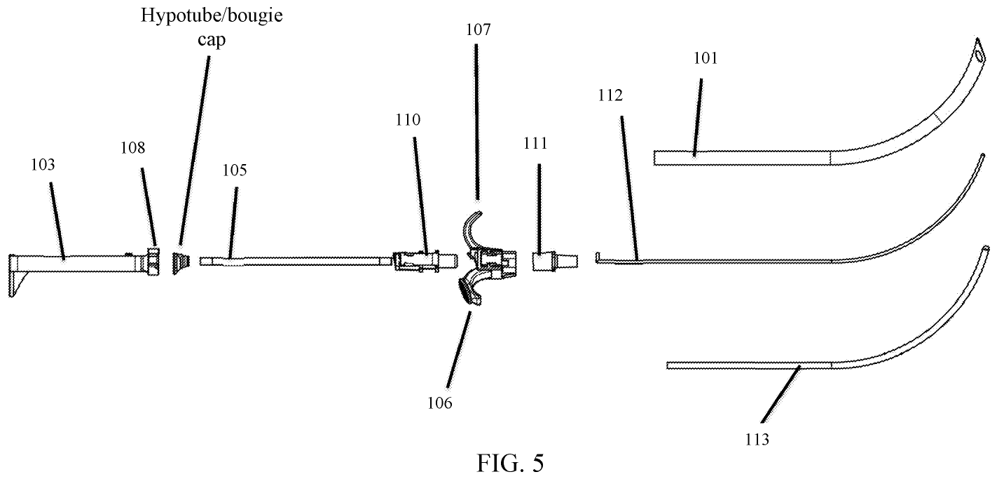

FIG. 5 is an exploded view of the assembly showing various components of one embodiment. The components comprising ETT advancer portion 103 (having a distal ETT collar portion 108 that engages the ETT connector), a bougie advancer portion not shown (operatively coupled to a bougie 113, stylet 112, or bougie and stylet assembly that can be positioned in the lumen of an ETT), a hypotube portion 105, at least two stop portions or finger rests—a first stop portion with a thumb rest 106 and a second stop portion 107. The stop portion of the handle can be removably or integrated with handle core 110. ETT connector 111 is removably or intergrated with the ETT tube and is the portion that is received by ETT collar 108. The hypotube portion 105 may be a rigid tube, made from any suitable biocompatible material known to one of ordinary skill in the art. Such materials may include, but are not limited to, rubber, silicon, plastics, stainless steel, metal-polymer composites, and metal alloys of nickel, titanium, copper cobalt, vanadium, chromium, and iron. In some embodiments, hypotube 105 may include layers of different materials and reinforcements such as braiding or coiling within the wall of hypotube 105.

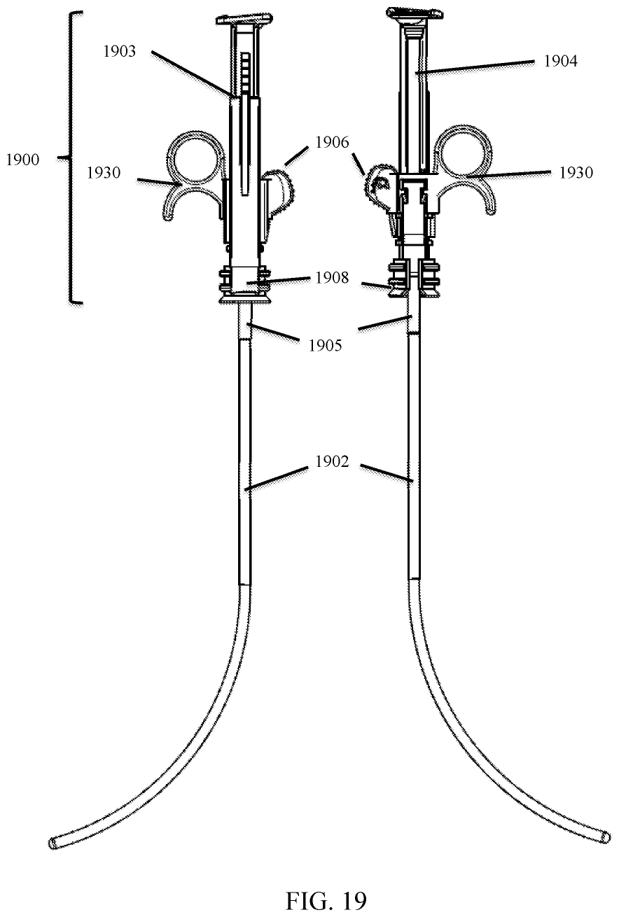

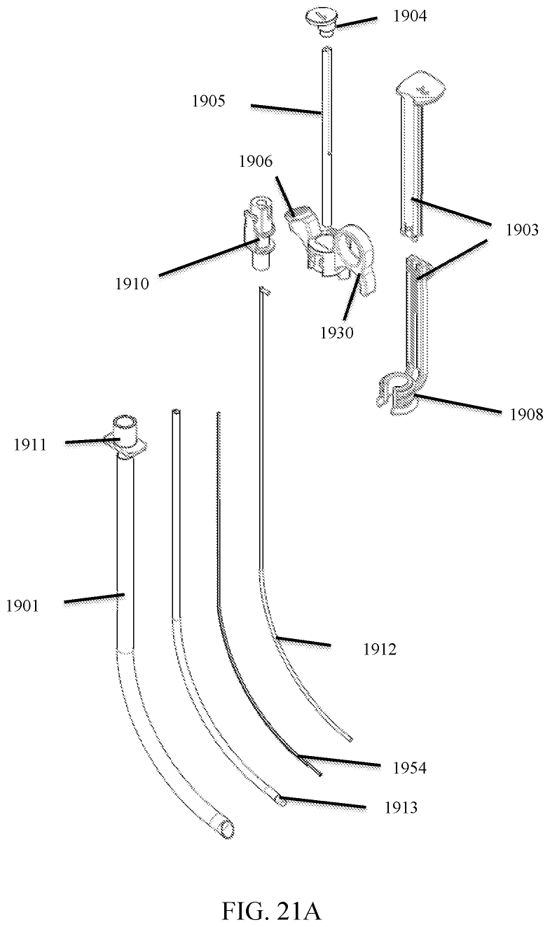

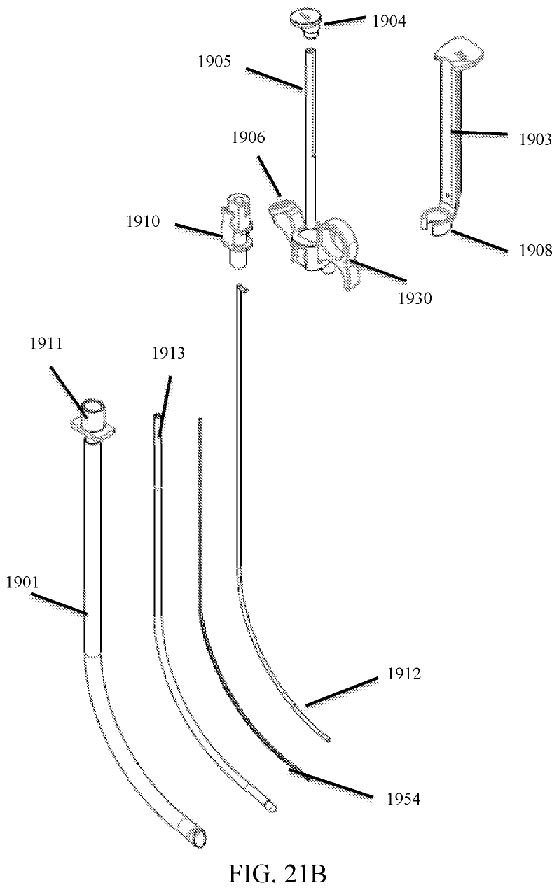

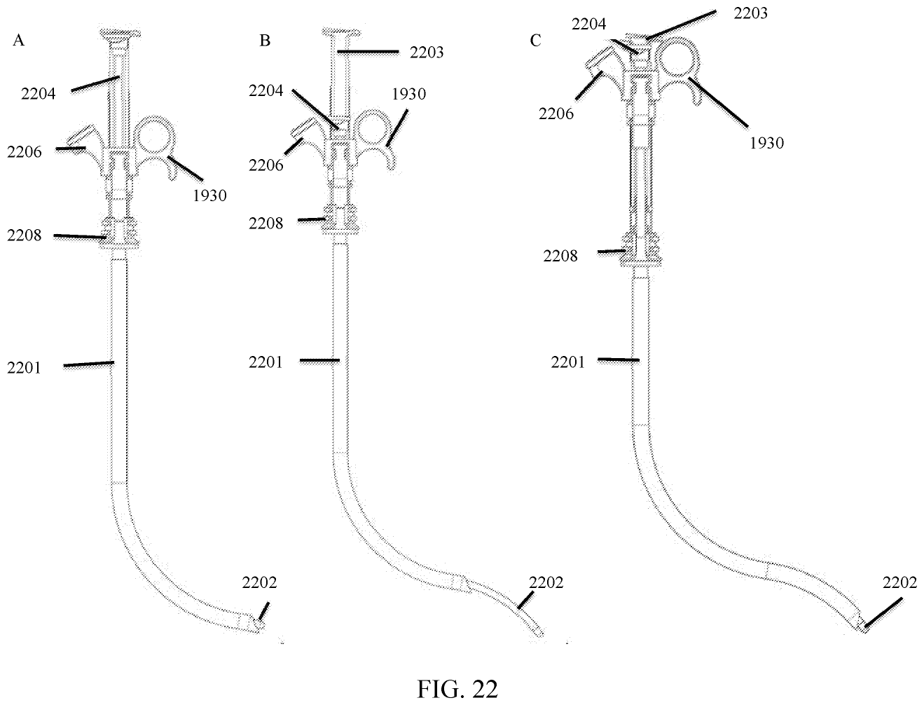

FIG. 19 to FIG. 21 illustrates an alternative embodiment for the device. With reference to the exploded illustration, FIG. 21, the handle can comprise an ETT advancer portion 1903, either an adjustable two piece (A) or a single size one piece (B) (having a distal ETT collar portion 1908 that engages the ETT connector), a bougie advancer portion 1904 (operatively coupled to a bougie, stylet, or bougie and stylet assembly that can be positioned in the lumen of an ETT), a hypotube portion 1905, at least two stop portions or finger rests—a first stop portion or finger rests with a thumb rest 1906 and a second stop portion 1930 configured with a loop forming a lumen providing for insertion of finger or thumb of the operator. It also allows an easy hand transition from either hold so the operator can perform the cannulation phase (bougie deployment followed by ETT advancement). The components comprising ETT advancer portion 1903 (having a distal ETT collar portion 1908 that engages the ETT connector), a bougie advancer portion 1904 (operatively coupled to a bougie 1913, stylet 1912, and/or a memory wire 1954 or bougie 1913 and stylet 1912 and/or memory wire 1954 assembly that can be positioned in the lumen of an ETT), a hypotube portion 1905 that can be operatively coupled to the bougie advancer portion and the bougie assembly, at least two stop portions or finger rests—a first stop portion with a thumb rest 1906 and a second stop portion 1907. The stop portion of the handle can be removably or integrated with handle core 1910. ETT connector 1911 is removably or integrated with the ETT tube and is the portion that is received by ETT collar 1908. The hypotube portion 1905 may be a rigid tube, made from any suitable biocompatible material known to one of ordinary skill in the art. Such materials may include, but are not limited to, rubber, silicon, plastics, stainless steel, metal-polymer composites, and metal alloys of nickel, titanium, copper cobalt, vanadium, chromium, and iron. In some embodiments, hypotube 1905 may include layers of different materials and reinforcements such as braiding or coiling within the wall of hypotube 1905.

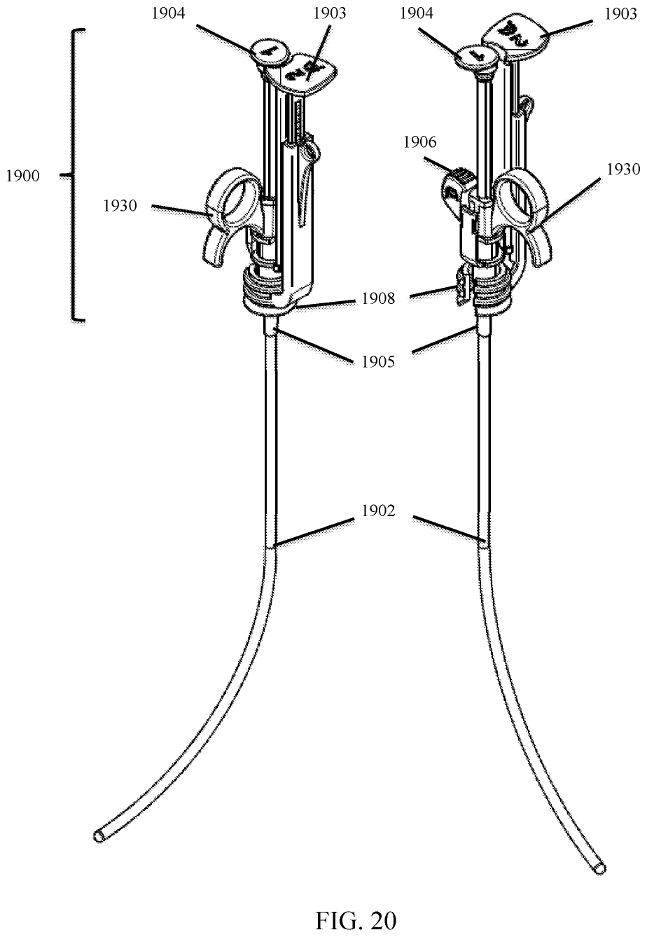

FIG. 19 and FIG. 20 shows an assembled example of one embodiment of a bougie-over-wire device assembly. FIG. 19 and FIG. 20 show handle mechanism 1900 configured to engaged with ETT (not shown) and having bougie 1902 configured to be positioned in the lumen of the ETT. This design allows the operator to hold ETT or handle during insertion. FIG. 20 shows a perspective view of handle mechanism 1900 having a bougie 1902 positioned, the bougie being designed to be positioned in the lumen of ETT. This design allows the operator to hold ETT or handle during insertion. Operator can actuate the bougie using bougie advancer 1904 and actuate ETT advancement via ETT advancer 1903. The design is also lower profile, allowing easier packaging and lower manufacturing costs. In certain instances, the operator can use stop portion/finger rest to assist in advancement of the ETT advancer or bougie advancer by engaging the stop portion/finger rest 1906, 1930 with the fingers, much like a hypodermic needle, to provide counter force to thumb applied force.

### A. ETT Advancer

With reference to FIG. 6, an ETT positioning mechanism can be incorporated into the ETT advancer component, such that the advancer is now comprised of multiple parts, an upper ETT advancer portion 120, a lower ETT advancer portion 121, ETT collar 108, and ETT advancer locking mechanism 122. An upper subcomponent 120 interacts with the handle, and a lower subcomponent 121 secures to the connector of the ETT. These two subcomponents interact in manner that allows the lower subcomponent to move upward or downward along the upper subcomponent in order to align and lock in the tip of the ETT at the appropriate position on the distal bougie-stylet complex in the device's resting state. The two subcomponents will interact via a sliding locking mechanism or a linear sliding ratcheting mechanism that can be adjusted, locked, or unlocked by the operator. FIG. 6A shows an adjustable ETT Advancer in mid-position. FIG. 6B. Shows upper subcomponent 120 and FIG. 6C shows lower subcomponent 121. FIG. 6D illustrates the adjustable ETT advancer in the most compact position. The ETT advancer can be adjusted to accommodate ETT of differing lengths. In certain aspects the locking mechanism can be a ratchet mechanism comprising a pawl 122 that engages or disengages notches or grooves 124.

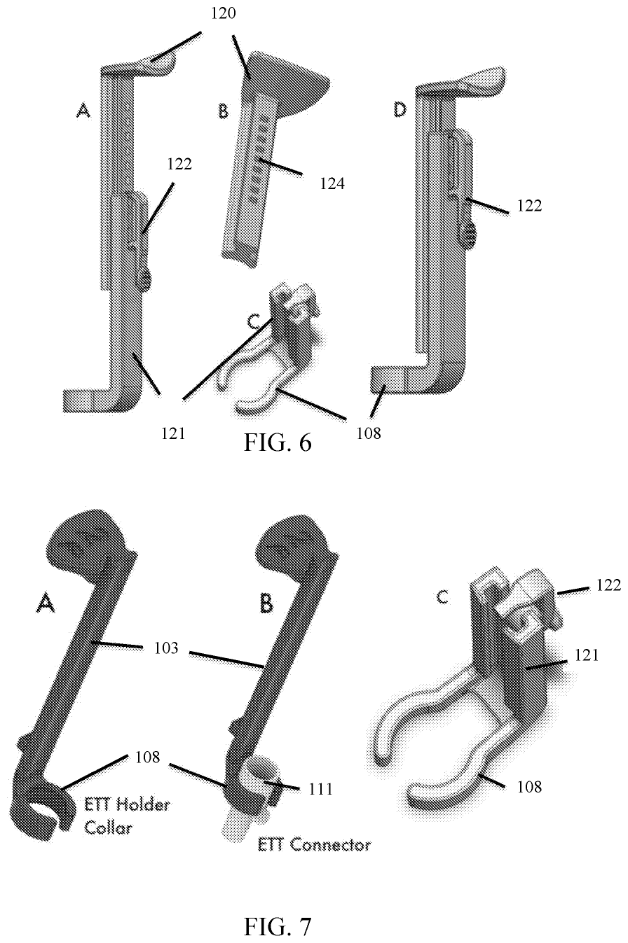

When the operator deploys the bougie, friction between the ETT and bougie pulls on the ETT which can pull the ETT off of the advancer collar and allow premature advancement of the ETT within the operational sequence. If the interaction is too loose, the ETT slides off prematurely. If the interaction is too tight, it becomes inordinately difficult for the provider to remove the ETT from the device. Through a proper combination of surface area, material selection, collar diameter, and collar shape ETT collar 108 will prevent the ETT connector from sliding off of the advancer prematurely during bougie deployment. FIG. 7A shows the ETT holder collar feature of an ETT advancer. FIG. 7B shows ETT holder collar 108 of the ETT advancer with ETT connector 111 inserted and held securely. FIG. 7C shows an example of an ETT holder collar feature associated with the lower adjustable ETT advancer portion.

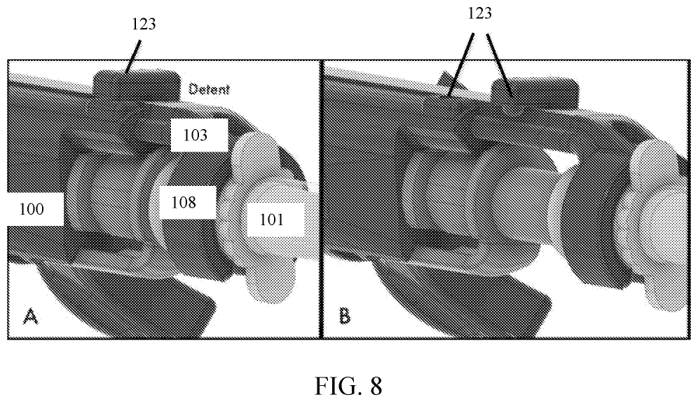

In certain aspects ETT connector 101 is securely affixed to the advancer collar 108, the advancer itself can move downward prematurely as the operator deploys the bougie. A locking mechanism 123 can be used to maintain the position of the advancer within the handle during bougie deployment by the use of a locking mechanism (e.g., a detent mechanism 123) between the advancer and the handle (FIG. 8). The locking or detent mechanism holds the advancer in place during bougie deployment, however the mechanism is capable of releasing the advancer when minimal pressure applied to the advancer thumb tab by the operator during the ETT advancement phase. FIG. 8 illustrates an example of a simple detent mechanism (ball type). FIG. 8A shows an ETT advancer in rest position with advancer-detent mechanism engaged. FIG. 8B shows an ETT advancer and detent disengaged from its resting position during the ETT advancement phase.

### B. Bougie-Wire-Handle Interface and the Hypotube

In certain embodiments an inner stylet wire (the stylet) can be connected to the handle either directly or through a central clip-in mechanism (FIG. 9 and FIG. 10). The stylet can have a proximal perpendicular bend or perpendicular inserted wire segment or peg. This necessitates a slot in the proximal bougie so it can slide over the wire. Since the bougie is already flexible, the slot further decreases its stability when an axial load is applied by the operator. A number of configurations of the device can be used to efficiently transmit an axial load to the bougie.

A partially slotted support shaft with a central channel (metal or plastic) that extends through the handle and connects to the bougie at some point below the handle. This is referred to as a hypotube 105 and can be constructed of metal or plastic. The hypotube will be capped with a button/thumb pad for efficient operation (FIG. 9). FIG. 9 illustrates a solid hypotube 105 that functionally stiffens the upper portion of the bougie 102 so bougie 102 will not bend when an axial load is applied by the operator. FIG. 9A shows a handle-bougie-hypotube-inner wire-handle core (clip-in) complex. FIG. 9B shows bougie-hypotube-inner wire-handle core complex. FIG. 9C shows a cross section of FIG. 9B demonstrating how wire/stylet 130 can be affixed the core of the handle 110 or handle core or clip-in. FIG. 9C also demonstrates the merger of the proximal bougie and the hypotube. The wire/stylet 130 can be bent as shown or have an inserted cross pin or peg. FIG. 9D-9E illustrate the hypotube 105-bougie 102-inner wire 130 complex in isolation.

A hollow, partially slotted support tube that surrounds the proximal aspect of the bougie and extends through the handle. This is also referred to as a hypotube and can be constricted of metal or plastic. In this form, the proximal aspect of the bougie exists all of the way to the top of the hypotube. The hypotube-bougie complex will be capped with a button/thumb pad for efficient operation (FIG. 10). FIG. 10 shows a hollow hypotube 105 that functionally stiffens the upper portion of the bougie 102 so bougie 102 will not bend when an axial load is applied by the operator. FIG. 10A shows a bougie 102-hypotube 105-inner wire 130-handle core 110 complex. FIG. 10B shows a cross section of 10A that also demonstrates how the wire 130 is affixed the core of the handle 110 or handle clip-in. The wire 130 can be bent as shown or have an inserted cross pin or peg. FIG. 10C-10E show hypotube 105-bougie 102-inner wire 130 complex. Note how the hypotube fully surrounds the bougie which is also slotted.

In other aspects a bougie with a differential shore hardness that is very rigid proximally and becomes softer distally such that it does not require a supportive hypotube can also be used. The bougie 102 can be connected to the hypotube 105 and capped with a button that will be at approximately the same level of the ETT advancer 103 button when the device is in the resting position.

Multiple intubation attempts can lead to increased complications. These operators may particularly benefit from the devices described herein. Further, the devices described herein may provide a particular benefit to operators outside-of-the-operating room during emergency tracheal intubation, and in austere conditions encountered by EMS personnel, military medics, and critical care air transport teams.

Certain embodiments are directed to an intubation assist device or intubation bougie and/or stylet. Particular embodiments include, but is not required to include, a handle as described herein. Other aspects may be, but are not required to be combined with the handle design or various stylet and bougie designs described herein.

Stylets and bougies that can be used in conjunction with the handle or a standard handle include hollow or solid stylets or bougies. In certain aspects the stylets can be rigid or semi rigid stylets. In certain aspects the stylet is configured to be positioned inside a lumen of a bougie. In other aspects the bougie is configured to be positioned inside the lumen of a hollow stylet. In still further aspects the distal region of the stylet can be modified. The distal region can be modified to include a variety of tips, lights, cameras, and or other functionalities. In certain aspects the handle can be modified to include a video screen or electronic monitoring components.

The handle can include an ETT advancer component. The ETT advancer component being configured to moveably connect to and/or hold the ETT during insertion as well as being capable of applying force to advance the ETT along the long axis of the stylet or bougie and into the trachea. In certain aspect the ETT advancer component is moveably connected by a track, groove, or the like. There may or may not be a ratcheting mechanism associated with the interaction between the handle body and the ETT advancement component.

In certain aspects the handle can incorporate a bougie that can be advanced to guide insertion of the ETT. The bougie can be advanced independently of the ETT or concurrently with the ETT. The tip of the bougie can be soft (“safe-soft”) and may or may not be malleable distally. The term “malleable” means that the section can be easily bent with the fingers and will retain its bent shape on its own without having to apply any external retaining force. Malleable is distinct from semi-rigid in that the amount of force needed to bend a malleable portion is less that than need to bend a semi-rigid portion.

In certain embodiments the handle is configured to be used with attachable/detachable stylets or bougies, with the stylets or bougies being disposable and the handle being reusable. In particular aspects the handle and the stylet/bougie are integrated and the whole device is disposable. In particular aspects the handle and the stylet/bougie are integrated and the whole device is reusable. In some instances, the device is configured to be used by one person during a tracheal intubation of a subject in that a bougie can be advanced and retracted and/or an ETT can be disengaged from the stylet/bougie by using the thumb of the hand holding the handle. In some instances, the device is configured to be used for tracheal intubation of a human. In some instances, the device is configured to be used for tracheal intubation of a non-human mammal subject.

The intubation assist devices described herein can be configured to be used in conjunction with a video laryngoscopy (VL) device, a direct laryngoscopy (DL) device, or a dual purpose flexible laryngoscopy device. Each of these configurations can be used in conjunction with a standard straight bougie tip, a malleable bougie tip, or an offset bougie tip. In certain aspects the tip is flexible and bends when encountering tissue in the larynx and trachea. In other aspects the tip maintains a memory or shape in that once positioned the tip can maintain the position of shape, e.g., an offset position. In still other aspects the tip can be permanently formed in an offset position.

The stylet can be a curved, hollow stylet incorporating an extendable bougie (e.g., a Design 1 bougie) positioned in the lumen of the stylet. This device will allow the operator to place the ETT, utilizing the stiffness of the stylet component, at or near the glottic aperture and advance the integrated bougie from the stylet through the glottis and into the trachea. Once the bougie is in place the ETT is advanced over the bougie into the trachea. In some instances, the hollow stylet provides the appropriate curvature in order to engage the glottic aperture during VL, and the bougie provides proper (tracheal) directionality for the ETT while force is applied to the ETT by the operator, e.g., via an ETT advancement component. The bougie also directs the ETT downward and away from the anterior larynx or anterior trachea where it can hang up or stall.

The term “hollow stylet” as used herein includes rigid or semi-rigid hollow stylet configured to have a bougie passed through the lumen of the stylet. In certain aspects a stylet has a thin tube configuration. The hollow stylet can have an external diameter of from 2.0, 2.5, 3.0, 3.5, 4.0, 4.5, 5.0, 5.5, 6.0, 6.5, 7.0, 7.5, 8.0, 8.5, 9.0, 9.5 to 10.0 mm, including all values and ranges there between, and an internal diameter of 1.0, 1.5, 2.0, 2.5, 3.0, 3.5, 4.0, 4.5, 5.0, 5.5, 6.0, 6.5, 7.0, 7.5, 8.0, 8.5, 9.0, to 9.5 mm, including all values and ranges there between.

The term “stylet” as used herein includes rigid or semi-rigid solid stylet can be configured to be positioned inside or in the lumen of a bougie. In certain aspects a stylet has a circular, oval, square, rectangular or other polygonal cross-section. The stylet can have a diameter of from 0.5, 1.0 2.0, 2.5, 3.0, 3.5, 4.0, 4.5, 5.0, 5.5, 6.0, 6.5, 7.0, 7.5, 8.0, 8.5, 9.0, 9.5 to 10.0 mm, including all values and ranges there between.

A “bougie” is a device used as a guide to aid insertion of other medical appliances (e.g., ETT) via the oral cavity or other potential anatomical space or opening. Typically, the bougie is removed once the medical appliance is in place. The length of a bougie can vary from 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0 21.5, 22.0, 22.5, 23.0, 23.5, 24.0, 24.5, 25.0, 25.5, 26.0, 26.5, 27.0, 27.5, 28.0, 28.5, 29.0, 29.5, 30, 35, 40, 45, 50, 55, 60, 65, to 70 cm, including all values and ranges there between. Bougie outer diameters can vary from 1.0, 1.5, 2.0, 2.5, 3.0, 3.5, 4.0, 4.5, 5.0, 5.5, 6.0, 6.5, 7.0, 7.5, 8.0, 8.5, 9.0, to 10 mm, including all values and ranges there between. The bougie may have a circular or elliptical cross section and can be made of a polymer such as aliphatic polyurethane, polytetrafluoroethylene, or other appropriate material. In certain aspects a bougie's flexibility or flexural modulus can changed along its length. For example the bougie can become more flexible as you move proximal to distal along its length. Each section having a specific flexural modulus, length and location along the bougie. For example the proximal end of the bougie can be more rigid than the distal end. A bougie can have a durometer in a range of about 20 Shore A to about 90 Shore A, as measured according to ASTM D2240. In certain aspects the flexible tip of a bougie can have a durometer from 20 shore A to 40 shore A. Other materials that can be used for the bougie include, but are not limited to, latex, silicon, polyester, nylon, rubber, and silk. In certain aspects the bougie material can comprise radiopaque or tracer material(s), such as barium sulphate. Specific examples of radiopaque materials include barioum diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosumetic acid, iotasul, iotetric acid, iothalamic acid, iotroxic acid, ioxaglic acid, ioxotrizoic acid, ipodate, meglumine, metrizamide, metrizoate, propyliodone and thallous chloride. The material may be a shape memory material and may be self-lubricating.

In some instances, the intubation stylet contains a rigid or semi-rigid curved, hollow or solid stylet with a proximal end and distal end; a flexible bougie with a handle, the bougie contained at least partially within the lumen of the hollow stylet and configured to be extended from and retracted into the distal end of the hollow stylet. The bougie handle can be configured to extend outside of the proximal end of the hollow stylet or be inserted into the hollow stylet up to a predetermined stop. In other aspects a stylet handle is attached to the proximal end of the hollow stylet. In other instances, the intubation stylet is coupled to a hollow endotracheal tube (ETT), wherein at least a portion of the hollow stylet is capable of being contained within or inserted into the lumen of the ETT, and wherein the ETT is capable of being extended past the distal end of the hollow stylet. The length and dimensions of the stylet can vary in relation to the length and dimension of the ETT. ETT internal diameters can vary from 2.0, 2.5, 3.0, 3.5, 4.0, 4.5, 5.0, 5.5, 6.0, 6.5, 7.0, 7.5, 8.0, 8.5, 9.0, 9.5 to 10 mm, including all values and ranges there between, to accommodate patients from pre-mature infants to adult males. The length of an ETT can vary from 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0 21.5, 22.0, 22.5, 23.0, 23.5, 24.0, 24.5, 25, 26, 27, 28, 29, 30, 31, 32, 33, 34 to 35 cm, including all values and ranges there between. The internal diameter of a hollow stylet can vary from 1.0, 1.5, 2.0, 2.5, 3.0, 3.5, 4.0, 4.5, 5.0, 5.5, 6.0, 6.5, 7.0, 7.5, 8.0, 8.5, 9.0, to 9.5 mm, including all values and ranges there between, and an external diameter of a hollow stylet can vary from 2.0, 2.5, 3.0, 3.5, 4.0, 4.5, 5.0, 5.5, 6.0, 6.5, 7.0, 7.5, 8.0, 8.5, 9.0, 9.5 to 10.0 mm, including all values and ranges there between. The length of a stylet from handle to distal end can vary form 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0 21.5, 22.0, 22.5, 23.0, 23.5, 24.0, 24.5, 25.0, 25.5, 26, 27, 28, 29, 30, 31, 32, 33, 34, 35, 36, 37, 38, 39, 40, 41, to 42 cm, including all values and ranges there between. In some instances, the hollow stylet curve is a distal curve with an angle of between about 10, 20, 30, 40, 50 and 60, 70, 80, 90, 100, 110, 120 degrees over the distal 5, 6, 7, 8, 9, 10, 11, 12, 13, 14, 15, 16, 17, 18, 19, or 20 cm, including all values and ranges there between, of the stylet. In some instances, the hollow stylet curve is a distal curve with an angle of about 80 degrees over the distal 5, 10, 15, or 20 cm of the stylet. In some instances, the hollow stylet curve is a distal curve with an angle of between about 30 and 55 degrees over the distal 5, 10, 15, or 20 cm of the stylet. In some instances, the hollow stylet curve is a distal curve with an angle of 45 degrees over the distal 5, 10, 15, or 20 cm of the stylet. In other instances, the hollow stylet is semi-rigid in that it is capable of being bent by a user using his/her hands without kinking the stylet prior to insertion, yet retain its shape during use of the device during a tracheal intubation procedure (i.e. , the stylet is semi-rigid). In certain aspects the degree of curvature is determined by the angle of elevation between the long axis and a second axis formed after the curve.

In another aspect, a method is disclosed for the tracheal intubation of a subject using any of the devices described herein. In some instances, the method includes one or more steps, such as obtaining or using any one of the tracheal intubation stylet devices as described herein, in certain aspects an ETT can be pre-loaded thereon; placing the distal end of the stylet device into and/or directly in front of or through the glottis of the subject; extending a flexible bougie past the distal end of the hollow stylet or placing the distal end of a solid stylet, through the vocal cords of the subject, and into the trachea; extending the ETT past the distal end of the stylet, through the glottis of the subject, and into the trachea; and removing the intubation stylet from the ETT. In some instances, the steps of the method are performed by one user. In some instances, the subject is a human. In some instances, the subject is a non-human mammal. One advantage to some of the intubation stylet designs described herein is that the advancement mechanism does not require the ETT incrementally advanced into the subglottis and trachea because the advance of the ETT by the mechanism allows the operator to gently and continuously advance the ETT into the trachea.

Certain embodiments can include a tip of contrasting color and a soft consistency (Safe-Soft) that prevents the ETT cuff from obscuring the operators view during VL, provides excellent visual acquisition of the tip, and prevents trauma to anatomic structures; or a tip that prevents ETT hang-up on the anterior, subglottic portion of the larynx/trachea during advancement of the complex into the proximal trachea.

In some instances the device includes a curved stylet comprising a proximal end and distal end, and a soft and flexible tip connected to the stylet, the tip extending past the distal end of the stylet and configured to extend past a distal end of a endotracheal tube (ETT) loaded on the device. In certain aspects the stylet is solid. In other aspect the stylet is rigid, In further aspects the stylet has a covering over the core of the solid and/or rigid stylet that extends beyond the distal end of the core forming a soft or pliable distal region. In other embodiments the solid stylet is positioned inside a lumen of a bougie or multiple lumen bougie. The distal region can be 1, 2, 3, 4, 5, to 5, 6, 7, 8, 9, 10 cm in length and taper into a rounded tip. In some instances, the distal region of the bougie or stylet is configured to extend 1 to 5 centimeters past a distal end of an ETT loaded on the device, or extend and terminate prior to reaching the end of the ETT. In some instances, the tapered tip is configured to extend 3.5 centimeters past the distal end of an ETT loaded on the device.

In some instances, the stylet or bougie is coupled to a hollow endotracheal tube (ETT), wherein at least a portion of the stylet or bougie is capable of being contained within the ETT, and wherein the ETT can be moved along the long axis of the stylet or bougie and extended past the distal end of the stylet or bougie during an intubation procedure. In certain aspects the ETT is a double lumen ETT. In other instances, the intubation stylet or bougie is coupled to a hollow endotracheal tube (ETT), wherein at least a portion of the stylet or bougie is capable of being contained within or inserted into the lumen of the ETT, and wherein the ETT is capable of being extended past the distal end of the stylet or bougie. The length and dimensions of the stylet or bougie can vary in relation to the length and dimension of the ETT. ETT internal diameters can vary from 2.0, 2.5, 3.0, 3.5, 4.0, 4.5, 5.0, 5.5, 6.0, 6.5, 7.0, 7.5, 8.0, 8.5, 9.0, 9.5 to 10 mm, including all values and ranges there between, to accommodate patients from pre-mature infants to adult males. The length of an ETT can vary form 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0 21.5, 22.0, 22.5, 23.0, 23.5, 24, 25, 26, 27, 28, 29, 30, 31, 32, 32, 33, 34, 35, 36, 37, 38, 39, 40, 41, or 42 cm, including all values and ranges there between. The diameter of a solid stylet or bougie can vary from 1.0, 1.5, 2.0, 2.5, 3.0, 3.5, 4.0, 4.5, 5.0, 5.5, 6.0, 6.5, 7.0, 7.5, 8.0, 8.5, 9.0, to 9.5 mm, including all values and ranges there between. The length of a stylet or bougie from handle to distal end can vary form 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0 21.5, 22.0, 22.5, 23.0, 23.5, 24.0, 24.5, 25.0, 25.5, 26, 27, 28, 29, 30, 31, 32, 32, 33, 34, 35, 36, 37, 38, 39, 40, 41, or 42 cm, including all values and ranges there between. In some instances, the stylet curve is a distal curve with an angle of between about 10, 20, 30, 40, 50 and 60, 70, 80, 90, 100, 110, 120 degrees over the distal 5, 6, 7, 8, 9, 10, 11, 12, 13, 14, 15, 16, 17, 18, 19, to 20 cm, including all values and ranges there between, of the stylet. In some instances, the stylet curve is a distal curve with an angle of about 80 degrees over the distal 5, 10, or 15 centimeters of the stylet. In some instances, the stylet curve is a distal curve with an angle of between about 30 and 55 degrees over the distal 5, 10, or 15 centimeters of the stylet. In some instances, the stylet curve is a distal curve with an angle of 45 degrees over the distal 5, 10, or 15 centimeters of the stylet. In certain instances, the stylet is rigid in that it is not capable of being bent by a user using his/her hands and retains its shape during use of the device during a tracheal intubation procedure. In certain instances, the stylet is malleable in that it is capable of being bent by a user using his/her hands yet retains its shape sufficiently during use of the device during a tracheal intubation procedure. In certain aspects the degree of curvature is determined by the angle of elevation between the long axis and a second axis formed after the curve.

In some instances, an intubation device is configured to be used by one person during a tracheal intubation of a subject. In some instances, the device is configured to be used for tracheal intubation of a human. In some instances, the device is configured to be used for tracheal intubation of a non-human subject.

Certain embodiments are directed to methods for tracheal intubation of a subject using a solid stylet. In some instances, the method includes: obtaining a stylet or bougie device disclosed herein with an ETT loaded thereon, the in certain aspects the stylet has a distal region that tapers to a rounded tip and placing the tapered tip through the vocal cords and into the trachea of the subject; advancing the distal end of the ETT through the vocal cords of the subject using the ETT advancer mechanism, and into the trachea. In some instances, the steps of the method are performed by one user. In some instances, the subject is a human. In some instances, the subject is a non-human.

In some instances, the device consists of a stylet or bougie handle with a thumb leverage point. The thumb leverage point can allow the operator to retract the stylet or bougie with their thumb as they advance the ETT through the vocal cords. In some instances the stylet handle is made of plastic and/or metal. In some instances, the hollow stylet or multi-lumen bougie emerges from the stylet handle and the hollow stylet is of metallic and/or plastic construction. In some instances, within the hollow stylet is a malleable bougie that can be easily advanced or retracted using a small bougie handle at the proximal end of the bougie. In certain aspects a stop is provided on the far proximal end of the bougie.

Non-limiting embodiments of an apparatus can include a hollow stylet coupled to a stylet handle and a bougie moveably coupled to the stylet. The stylet handle can have a member perpendicular to the long axis of stylet and a thumb leverage point that is configured to receive a force applied by the users thumb to separate stylet from an ETT during ETT insertion. In one embodiment configured for video laryngoscopy, the device has a slow, elongated distal bend of approximately 60, 70, to 80 degrees. Another embodiment is configured for direct laryngoscopy, the device that can have a shorter curved portion of approximately 45 degrees. Yet another embodiment is configured to enable modification of the hollow stylet portion by bending a distal portion of the stylet to facilitate intubation during VL or DL. A region of the stylet can be made from a semi-rigid material that can be bent to a desired curvature and maintain that curvature once bent.

Non-limiting embodiments can include a handle. An handle has a long axis running perpendicular to the long axis of the stylet. The device can include a stylet, e.g., a hollow stylet or solid wire stylet, coupled to the handle and a bougie moveably positioned around the stylet or in the lumen of the stylet. The handle can have at least two stop portions or finger rests that extend from the body of the handle. A posterior stop portion or finger rests can have a thumb rest that is used during the insertion phase of tracheal intubation. The thumb rest can provide a thumb leverage point that is configured to receive a force applied by the users thumb during manipulation of the device while it is being grasped around the handle/ETT assembly. The bottom of the stop portions or finger rests are configured to rest on palmar aspect of a user's fingers after the operator has transitioned the right hand for the cannulation phase of tracheal intubation which involves first deploying the bougie into the trachea and then advancing ETT into the trachea. The handle can be configured with an ETT advancer and bougie advancer mechanism. The ETT advancer is configured to engage an ETT to be advanced in to the trachea by providing a proximal thumb tab for advancement of the ETT using pressure applied by the thumb. There can be at least one thumb stop on the proximal portion of the advancer. The distal portion of the advancer can be configured to connect to the ETT (ETT connector or holder portion). In certain aspects the ETT advancer is connected by a track configured to guide the ETT along the stylet. There ETT advancer may be comprised of multiple sections and there may or may not be an adjustable ratcheting or locking mechanism associated with the ETT advancer. The stop portions or finger rests of the handle can be in an offset configuration in order to optimize handle ergonomics. The offset can be measured as an angle formed between the long axis of the stop portions. In certain aspects the handle can have an optional extendable bougie. If present, the bougie may or may not have a “safe-soft” tip and may or may not be malleable distally. In certain embodiment the bougie comprises a shape memory portion.

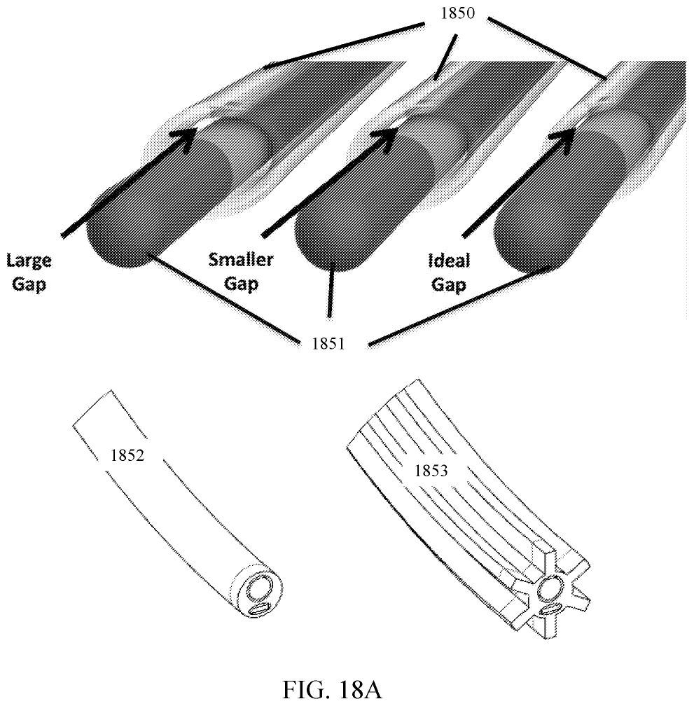

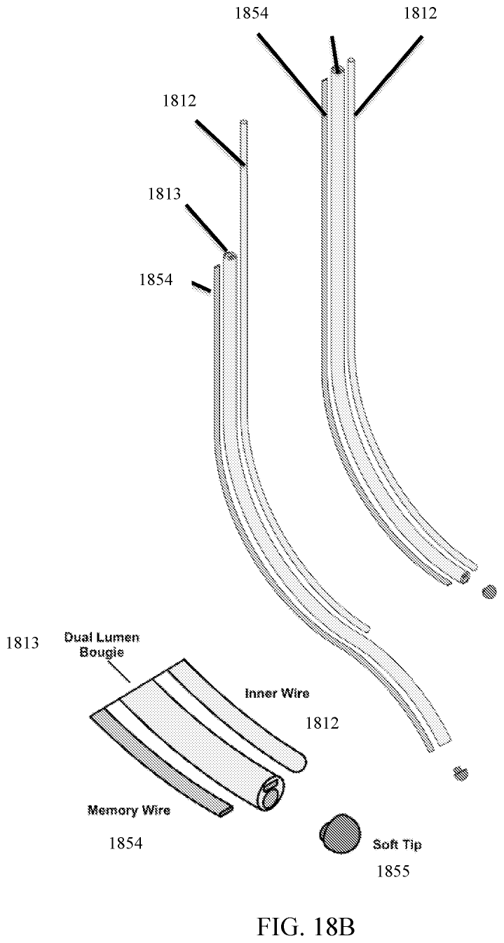

In certain aspects, the bougie outer diameter (OD) and overall bougie length can be matched to different sized ETT's. The reason for this is that the inner diameter (ID) of the respective ETT's is to match or be compatible the OD of the bougie in order to minimize the gap between ETT and bougie in order to (a) avoid ETT hang up on the glottic aperture (see FIG. 18), and (b) to further lessen trauma and friction on the anterior trachea during ETT deployment, and (c) serve as an overall better guide for the ETT. From an engineering point of view, this will require a very low friction bougie. This will also allow more length specific bougies for the different sized ETT's because their overall length (OAL) is different for each size. However, there is fairly significant variability amongst manufacturers, so the advance adjustment mechanism can be used when needed. The gap will be somewhere between 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, to 2.0 mm, including all values and ranges there between. In a particular aspect the gap is or is between 0.5-0.7 mm.

In certain aspects, friction between the ETT and bougie can be reduced by using extrusions of smooth surfaced bougies (e.g., see 1852 of FIG. 18), or longitudinal ribbed design (e.g., see 1853 of FIG. 18). In certain aspects, bougies will be matched with a hypotube using an adapter segment where a hypotube will have a distal diameter to match the OD of the bougie and connect the bougie to the hypotube that can be made having either a diameter to match the bougie or a constant diameter that is coupled with bougies of different diameter via the adapter.

In one embodiment the stylet can elongate to a slow, distal bend of approximately 20, 30, 40, 45, 50, 60, 70, to 80 degrees, including all values and ranges there between. In another embodiment the stylet can be configured for direct laryngoscopy, the device that can have a shorter curved portion of approximately 20, 30, 40, 45, 50, 60, 70, to 80 degrees, including all values and ranges there between. In yet another embodiment configured to enable modification of the hollow stylet portion by bending a distal portion of the stylet to facilitate intubation during VL or DL. The distal region can be made from a semi-rigid material that can be bent to a desired curvature and maintain that curvature once bent.

In various embodiments a bougie can have a “safe-soft” tip that may or may not be malleable distally.

In certain aspect a bougie described herein can be positioned within or external to a stylet and is configured to be advanced or retracted. In certain aspects the bougie is configured to have a stylet positioned in a lumen of the bougie. A bougie may or may not comprise a rod or gum elastic material which combines stiffness with flexibility at body temperatures. In some instances, the bougie is very soft at its most distal end having a “Safe-Soft” tip (e.g., the distal 2, 3, 4, 5, or 6 cm of the bougie can be a soft tip). In certain embodiments the “Safe-Soft” tip is essentially straight or has a terminal portion that is in an offset configuration. The soft tip can minimize the possibility of airway injury during advancement of a bougie. In some instances, the distal end of the bougie is slightly bulbous so it cannot be retracted fully into a hollow stylet by the operator, nor can it be pushed back into the stylet during the intubation procedure. In some instances, the bougie has a small handle or stop or button at the proximal end that the user or an assistant can push to advance the bougie. A stop can also serve to prevent excessive advancement of the bougie into the patient or loss of the bougie into the ETT or the patient.

Certain embodiments of the device can be used during video laryngoscopy. The insertion of the ETT using a stylet can be with the assistance of a video or direct laryngoscope. Operator(s) can steer ETT towards the glottic aperture, direct soft-tipped bougie and ETT tip through the glottis, and glide bougie followed by the ETT into the trachea with unparalleled ease. The devices described herein can help overcome the challenge encountered in advancing ETT into the trachea during VL despite an adequate view of the vocal cords. The devices can also facilitate VL or DL intubation during less than optimal VL OR VL views. Therefore, some embodiments of the device disclosed herein can be used to facilitate tracheal intubation during direct laryngoscopy (DL) under less than ideal intubating conditions. The devices decried herein can be of particular benefit to operators outside-of-the-operating room during emergency tracheal intubation, and in austere conditions encountered by EMS personnel, military medics, and critical care air transport teams.

In some instances, the device can be used on a human subject, a non-human mammal subject, or a non-mammal animal subject.

In certain embodiments the stylet device described herein can be included in a pre-sterilized medical procedure kit and used for various medical procedures. In certain aspects sterilized procedure kits are provided with a plurality of components used in connection with a particular medical procedure. Certain embodiments are directed to sterilized kits to maintain a sterile environment or reduce the risk for infection during a procedure. Any materials that will be in contact with the patient can be provided in sterile compartments or packaging that can be opened just prior to use in order to maintain sterility or reduce contamination.

The stylet and bougie devices described herein can be used for VL and DL inside or outside the operating room setting. VL use has particularly expanded in the settings of out-of-the-operating room and out-of-hospital tracheal intubation. In these settings, non-anesthesiology personnel are usually the operators, and they have varying degrees of airway management skill and experience. These operators may particularly benefit from a device like those described herein. Therefore, the devices can be used in the operating room, emergency room, intensive care units, on location medial emergencies by EMS/Fire units, military field and air transport applications.

Unless excluded, all elements and description from each embodiment can be used in conjunction with other described embodiments, e.g., element 104 can be equivalent or similar to 1904, etc.

### II. Bougie Designs

One embodiment is designated “Design 1”. Design 1 includes a stylet with steered inner bougie having a shape memory portion. The term “shape memory” refers to materials capable of recovering from a deformation and returning to a default geometry. Materials with shape memory include, but are not limited to titanium, nickel, nitinol, stainless steel alloys, niobium, zirconium, cobalt-chrome alloys, molybdenum alloys, tungsten-rhenium alloys and any combinations thereof. The bougie leads, then ETT follows. Design 1 is a bougie within a stylet; however, Design 1 incorporates a segment or portion having shape memory (e.g., a curved portion that can be deformed to fit through a stylet) within the distal portion of the bougie. This component of the bougie may extend for its entire length but only the distal portion has shape memory. The shape of the shaped portion bends or is in a direction opposite of the stylet curve, so that as the bougie deploys it curves downward or opposite the curve of the stylet. In certain aspects the shaped portion of the bougie can be at most, at least, or about 0.5, 1, 2, 3, 4, 5, 6, 7, 8, 9, 10, 11, 12, 13, 14, 15 mm in length, including all values and ranges there between. In other embodiments the ratio of bougie length to the shaped portion is about 50, 45, 40, 35, 30, 25, 20, 15, 10, 8, 6, 4 to 1, including all ratios and ranges there between. In certain aspects the shaped portion can be a wire or flat strip. The diameter or cross-sectional area of the shaped portion can vary along its length. The shaped portion can bend at least or about 0.5, 1, 2, 3, 4, 5, 6, 7, 8, 9, 10, 20, 30, 40, 50 degrees, including all values and ranges there between.

Shape memory materials include, but are not limited to titanium, nickel, nitinol, stainless steel alloys, niobium, zirconium, cobalt-chrome alloys, molybdenum alloys, tungsten-rhenium alloys and any combination thereof. In certain aspects the shape memory material is nitinol. In certain aspects the activation temperature of the nitinol can vary, but is usually well below that of ambient temperature such that is shape-maintaining before and after interaction with the patient.

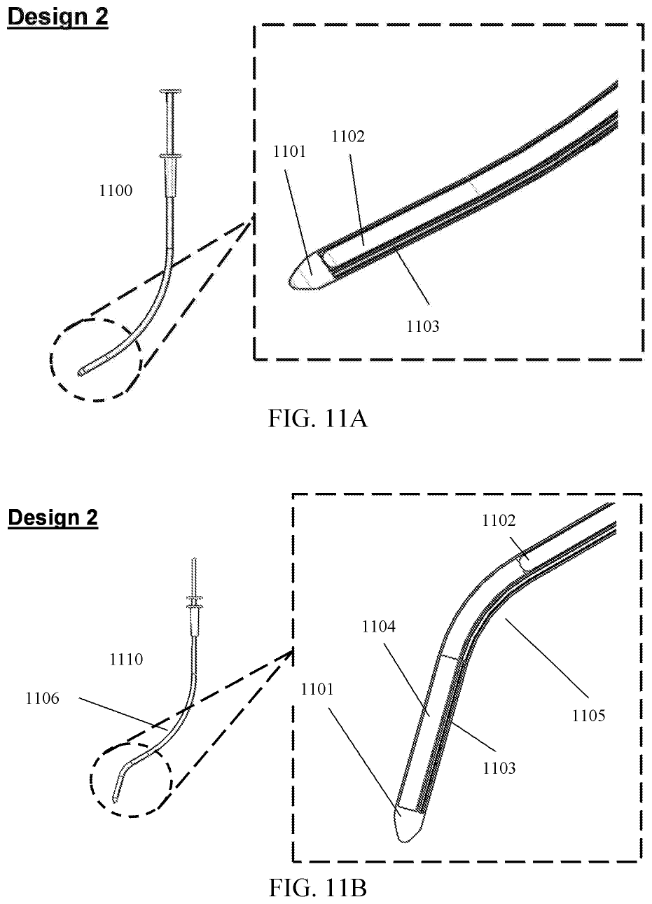

Referring to FIG. 11A, 11 B, 14, and 15. Other embodiments are designated “Design 2.” Design 2 is a bougie-over-stylet (inner wire) design. The rigidity and shape of the bougie is provided by an inner wire or stylet. The stylet may be pre-formed and rigid, or semi-malleable in that the distal bend can be adjusted by the operator's hands, yet the stylet maintains shape during the tracheal intubation procedure. After insertion of the ETT & bougie tip at or into the glottis, the sequence of operation begins with shape memory material-guided bougie deployment into the trachea followed by ETT advancement into the trachea which are accomplished via separate actuators on the handle of the stylet. In certain aspects the bougie is a two-lumen extrusion that allows for the stylet to sit in one lumen and a guide including a shape memory portion (“guide”) to sit in the other lumen. The shape memory guide directs the bougie to take the reverse curve during bougie deployment. This component of the bougie may extend for the bougie's entire length but only the distal portion has shape memory. The shape memory guide is made of metal (titanium, nickel, nitinol, stainless steel alloys, niobium, zirconium, cobalt-chrome alloys, molybdenum alloys, tungsten-rhenium alloys and any combination thereof. In certain aspects the shape memory portion is nitinol). The user can push the handle actuator to deploy the bougie and shape-memory guide within the bougie simultaneously. At some point the shape memory guide within the bougie stops moving due to a stop in the handle, and the bougie continues downward into the trachea and comes to rest in the mid trachea (FIG. 11B, 14, 15). In another version, the shape memory guide within the bougie continues with the bougie until the bougie comes to a stop when its actuator button hits the handle, and the bougie comes to rest in the mid trachea. In both versions, the operator then deploys the ETT over the bougie using the ETT advancer thumb tab.

Referring to FIG. 11A the bougie/stylet assembly is in its non-deployed (resting) state 1100. The non-deployed bougie consists of three components; the multi lumen bougie extruded housing with round tip 1101, the inner stylet 1102 and the shape memory guide 1103. Referring to FIG. 11B the bougie assembly is in its deployed state 1110. The multi lumen bougie 1101 extends past the inner stylet 1102 leaving a void 1104. The shape memory guide extends with the bougie guiding it in an opposing direction 1105 of the original radius 1106.

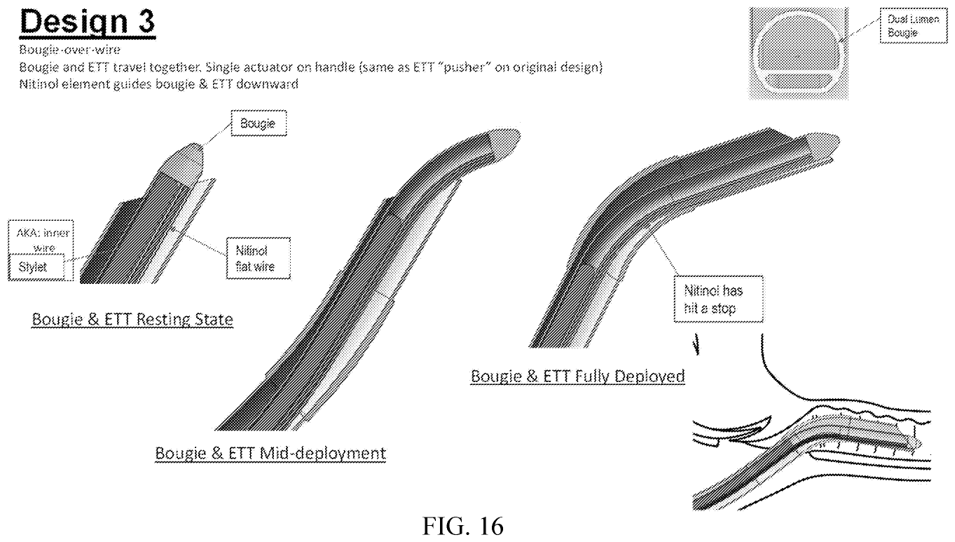

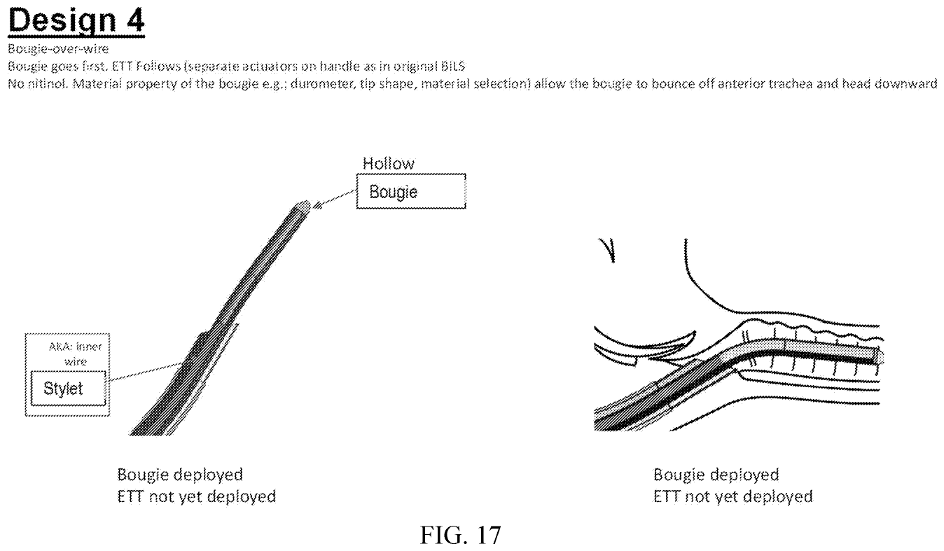

Referring to FIG. 16 Certain embodiments are designated as “Design 3”, which is a bougie-over-stylet design. Bougie and ETT move together. The shape memory guided bougie bends and guides the ETT as both move downward together. The bougie can be a two-lumen extrusion that allows for the stylet to sit in one lumen and a shape memory guide to sit in the other lumen. The shape memory guide guides or directs the Bougie to take the reverse curve during bougie deployment. In certain aspects the Inner stylet is made of metal (aluminum, steel, etc.).

The user pushes on the handle actuator to deploy the bougie and shape memory guide and the ETT together. In this configuration, there is only one actuator on the handle that deploys the ETT and the bougie together. As in Design 2, the bougie is a two-lumen extrusion that allows for the stylet to sit in one lumen and a shape memory guide to sit in the other lumen. The shape memory guide guides or directs the bougie to take the reverse curve during deployment. This component of the bougie may extend for the bougie's entire length but only the distal portion has shape memory. At some point the shape memory guide within the bougie stops moving due to a stop in the handle, and the bougie continues downward into the trachea along with the ETT. In another version, the shape memory guide within the bougie continues with the bougie until the bougie comes to a stop when the advancer button stops at the handle, and the bougie & ETT come to rest simultaneously in the mid trachea.

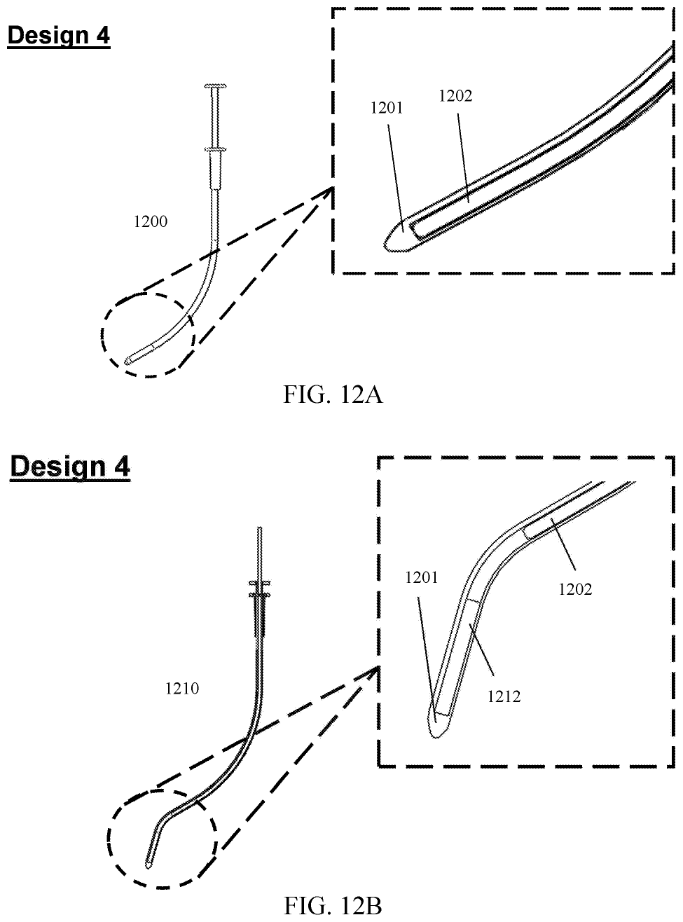

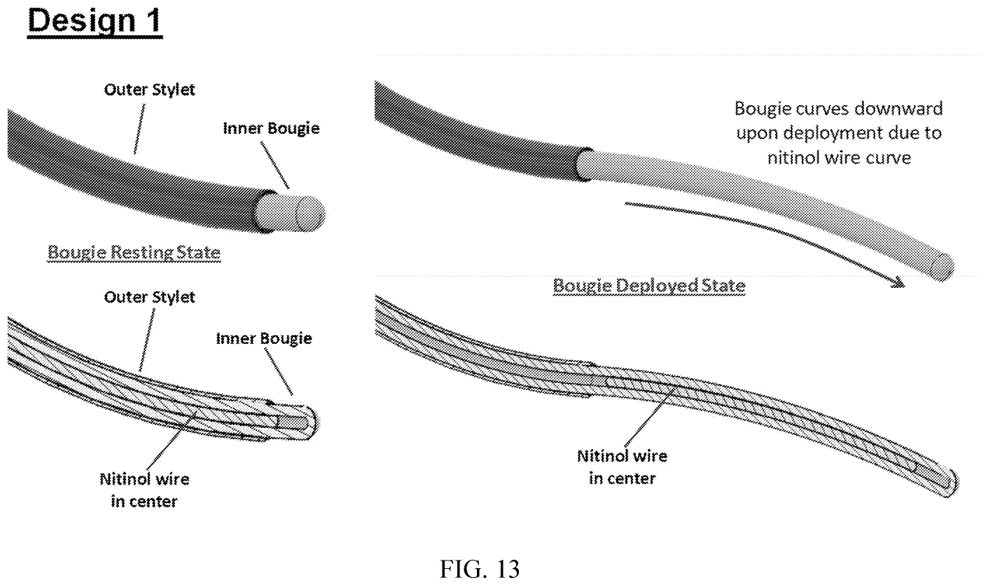

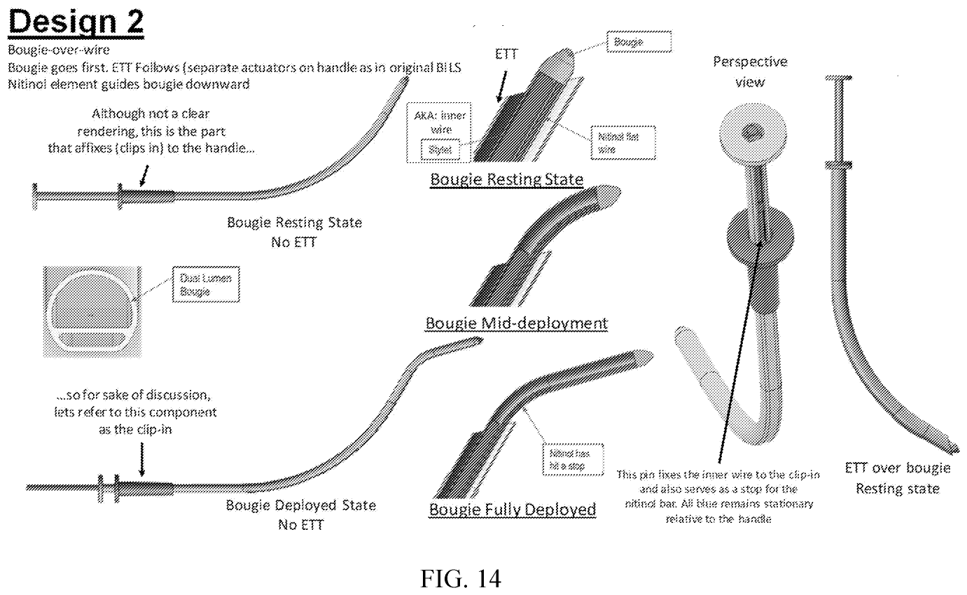

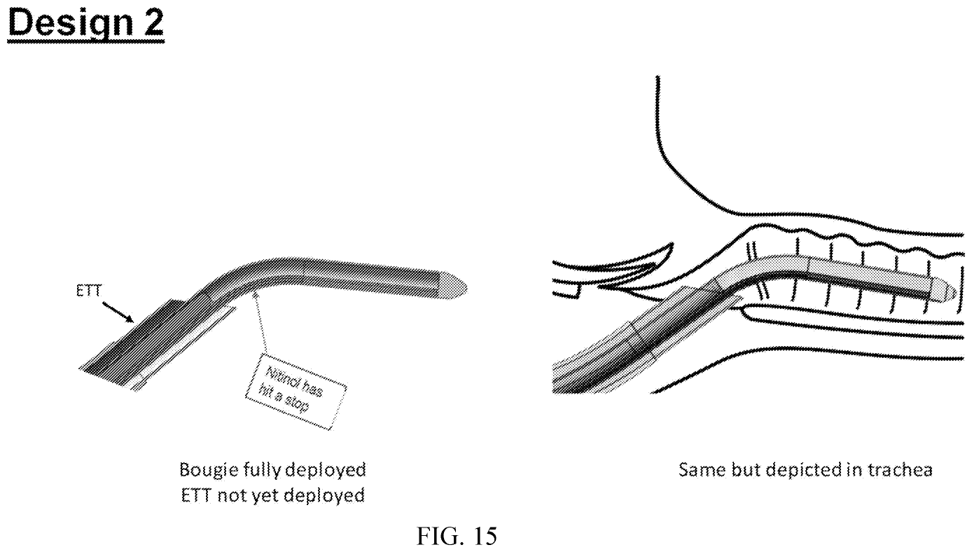

Other embodiments are designated as “Design 4”, which is a bougie-over-stylet design. Bougie leads then ETT follows via separate actuators on the handle. No shape memory portion is involved. The bougie is not guided downward. It simply bounces off the anterior trachea because the distal portion is soft. Referring to FIG. 12A the bougie assembly in the un-deployed state 1200 contains an outer single lumen bougie 1201 and an inner stylet 1202. Referring to FIG. 12B the bougie assembly in the deployed state 1210 contains an outer single lumen bougie 1201 and an inner stylet 1202. When deployed the inner stylet stays in its original position and the outer bougie advances creating a gap 1212 allowing the outer bougie 1201 to be more malleable and move past the tracheal rings.

1. A tracheal cannulation device comprising:
a curved, stylet comprising a proximal end and distal end;
a flexible bougie positioned in the lumen of the stylet or surrounding the stylet;
a handle attached to the proximal end of the stylet, the handle having
(i) a handle core portion forming a lumen or opening for a stylet or bougie to traverse the handle and at least two stop portions or finger rests projecting from the handle core portion away from the long axis of the device, a first, posterior stop portion comprising a thumb rest,
(ii) an endotracheal tube (ETT) advancer portion having a proximal thumb tab and distal ETT collar,
(iii) a bougie advancer portion having a proximal thumb button coupled to the flexible bougie; and
a rigid hypotube traversing the handle and forming a lumen in which the bougie is positioned and to which the stylet is attached.

2. The device of claim 1, wherein the ETT advancer portion is an adjustable two-piece ETT advancer portion.

3. The device of claim 1, wherein the handle further comprising a locking mechanism to reversibly secure the ETT advancer to the handle core.

4. The device of claim 3, wherein the locking mechanism is a detent or ratchet mechanism.

5. The device of claim 1 or 2, further comprising a hollow endotracheal tube (ETT), wherein at least a portion of the stylet or bougie is capable of being contained within the ETT, and wherein the ETT is capable of being extended past the distal end of the stylet.

6. The device of any of claims 1 to 5, wherein the stylet curve is a distal curve with an angle of between 20 and 90 degrees.

7. The device of any of claims 1 to 5, wherein the stylet curve is a distal curve with an angle of between 30 and 55 degrees.

8. The device of any of claims 1 to 7, wherein the device is configured to be used for tracheal intubation of a human.

9. The device of any of claims 1 to 7, wherein the device is configured to be used for tracheal intubation of a non-human mammal subject.

10. A bougie comprising:
a proximal end and distal end, wherein the proximal end is configured to be coupled to a deployment device, and the distal end comprising a rounded tip configured to deploy into the trachea of a subject; and
the bougie having a guide that comprises a shape memory portion that returns to a default geometry that curves away from the curve of a deployment device when the bougie is deployed during use.

11. The bougie of claim 10, wherein the shape memory portion if 1 to 18 mm in length.

12. The bougie of claim 10, wherein the shape memory portion is titanium, nickel, nitinol, stainless steel alloys, niobium, zirconium, cobalt-chrome alloys, molybdenum alloys, tungsten-rhenium alloys and any combination thereof.

13. The bougie of claim 10, wherein the shape memory portion is nitinol.

14. The bougie of claim 10, wherein the guide has an outer coating.

15. A bougie comprising:
a proximal end and distal end, wherein the proximal end is configured to be coupled to a deployment device, and the distal end comprising a rounded tip;
at least two lumens, (a) a first lumen containing an inner stylet wherein the bougie is configured to extend beyond the stylet, and (b) a second lumen containing a shape memory guide that is configured to extend with the bougie, the shape memory guide comprising a shape memory portion that returns to a default geometry that curves away from the curve of the stylet when the bougie is extended beyond the stylet.

16. The bougie of claim 15, wherein the shape memory portion is nitinol shape memory portion.

17. The bougie of claim 16, wherein the shape memory portion is a wire or a strip.

18. The bougie of claim 15, wherein the stylet is a rigid stylet.

19. The bougie of claim 15, wherein the stylet is an aluminum or stainless steel stylet.

20. The bougie of claim 15, wherein the stylet is a curve with an angel of between 20 and 90 degrees.

21. The bougie of claim 15, wherein the stylet is capable of being bent by a user and retain its shape during use of the device during a tracheal intubation procedure.

22. The bougie of any of claims 15 and 18, further comprising a hollow endotracheal tube (ETT), wherein at least a portion of the bougie is capable of being contained within the ETT, and wherein the ETT is capable of being extended past the distal end of the bougie.

23. A bougie comprising:
a proximal end and distal end, wherein the proximal end is configured to be coupled to a deployment device, and the distal end comprising a rounded tip configured to deploy into the trachea of a subject, wherein the bougie is hollow and configured to bend when impacting tracheal wall during deployment.

24. A method for tracheal intubation of a subject having a glottis aperture, vocal cords, and a trachea, the method comprising:
obtaining a bougie of any of claims 10 to 23 with an ETT loaded thereon;
placing the distal end of the bougie into and/or directly in front of the glottic aperture of the subject;
extending the bougie past the distal end of the ETT, through the vocal cords of the subject, and into the trachea;
extending the ETT past the distal end of the bougie, through the vocal cords of the subject, and into the trachea; and
removing the bougie from the ETT.

25. The method of claim 24, wherein the subject is a human.

26. The method of claim 24, wherein the subject is a non-human mammal.

27. A method for tracheal intubation of a subject having a glottic aperture, vocal cords, and a trachea, the method comprising:
obtaining a bougie of claim 15 with an endotracheal tube (ETT) loaded thereon;
placing the distal end of the ETT/bougie into and/or directly in front of the glottic aperture of the subject;
extending the ETT/bougie through the glottis where the bougie bends when in contact with the trachea guiding the ETT into the trachea; and
removing the bougie from the ETT once the ETT is positioned in the trachea.
